(19) United States
(12) Patent Application Publication
(10) Pub. No.: US 2024/0210371 A1
(43) Pub. Date: Jun. 27, 2024

(54) SYSTEM FOR THE ELECTRO-PHYSIOLOGICAL ANALYSIS OF ONE OR MORE PLANTS

(71) Applicant: Fabian Le Bourdiec, Bordeaux (FR)

(72) Inventors: Fabian Le Bourdiec, Bordeaux (FR); Patrice Cocheteux, Bordeaux (FR)

(21) Appl. No.: 17/791,368

(22) PCT Filed: Jan. 4, 2021
(86) PCT No.: PCT/EP2021/050015

§ 371 Date: Oct. 3, 2023

(30) Foreign Application Priority Data
Jan. 7, 2020 (FR) 2000094

(51) Int. Cl. G01N 33/00; G01N 27/02

(52) U.S. Cl. CPC G01N 33/0098; G01N 27/028

(57) ABSTRACT

The invention relates to a system (1, 10, 20, 30) for the electrophysiological analysis of one or more plants (P), comprising a first acquisition device (2) comprising a plurality of first electrodes (E1) each intended to be connected to the same plant or to a separate plant to pick up a first analog electric potential (P1), the first acquisition device comprising a casing (21) in which a multi-channel analog-digital converter (3) is arranged that is provided with a plurality of first inputs (31) each connected to one of the first electrodes to receive one of the first analog electric potentials, the multi-channel analog-digital converter being arranged to measure the potential difference (P21) between each first analog electrical potential received and a reference electric potential (P2, 7) and to convert each measured potential difference into a digital signal (S21).

The invention relates to the field of electrophysiological analysis of plants. More specifically, the invention relates to a system for the electrophysiological analysis of one or more plants.

It has been observed that plants emit electrical signals, particularly in the form of action potentials, reflecting their physiological state. By measuring these electrical signals via electrodes and analyzing them, it is thus possible to determine the physiological state of a plant more rapidly and more reliably than by means of direct observation. In addition, if stress or illness is detected, it is also possible to propose corrective actions immediately and automatically to remedy it.

However, existing systems for the electrophysiological analysis of plants are designed for laboratory use and are not suitable for agricultural use, in which they would be subject to severe constraints compared to those of laboratories, and in particular in terms of tightness and robustness with respect to climatic conditions, which may lead to their deterioration or destruction. Furthermore, since electrophysiological analysis for agricultural use is based on statistical methods, it is necessary to obtain a plurality of electrical signals simultaneously, over long periods of time. However, the known systems are designed to capture and analyze the electrical signals of just one plant at a time. These systems are therefore not compatible with agricultural use, in particular when harvesting and signal analysis must be carried out in real time.

The present invention is situated in this context and thus aims to remedy the drawbacks mentioned, by proposing a system for the electrophysiological analysis of one or more plants that is compatible with agricultural use and that makes it possible to collect data relating to a set of electrical signals emitted simultaneously by this or these plants.

For these purposes, the subject of the invention is a system for the electrophysiological analysis of one or more plants, comprising a first acquisition device comprising a plurality of first electrodes each intended to be connected to the same plant or to a separate plant to pick up a first analog electric potential, the first acquisition device comprising a casing in which a multi-channel analog-digital converter is arranged that is provided with a plurality of first inputs each connected to one of the first electrodes to receive one of the first analog electric potentials, the multi-channel analog-digital converter being arranged to measure the potential difference between each first analog electrical potential received and a reference electric potential and to convert each measured potential difference into a digital signal.

It is thus understood that the invention makes it possible, owing to the multichannel analog-digital converter, to simultaneously pick up a plurality of electrical signals emitted by the plant(s) and to determine, from these signals, differences in electric potential, which are converted into digital signals that can be analyzed, immediately or later. In addition, integrating the multi-channel analog-digital converter into a casing allows the analysis system to withstand the various environmental constraints of the agricultural environment.

Advantageously, the casing comprises a plurality of connectors each able to receive one of the first electrodes and arranged to transmit said first analog electric potential picked up by said first electrode to one of the first inputs of the multi-channel analog-digital converter. Where appropriate, the casing is arranged to be tight.

If desired, each first electrode is a needle-type electrode. This type of electrode is particularly suitable for agricultural use, the connection of a needle-type electrode to the plant being more robust than connections with a cup-type electrode in a context where plants are likely to grow and undergo mechanical stresses, in particular due to the wind.

Advantageously, the first acquisition device comprises at least one electric power source arranged to power the multi-channel analog-digital converter. If necessary, the electric power source is arranged in the casing. This feature allows the acquisition device to be rendered autonomous, so that it can pick up electrical signals emitted by the plant(s) for long periods of time. Alternatively or cumulatively, the casing of the first acquisition device may comprise a connector capable of receiving an external electric power supply.

If desired, each first input connected to one of the first electrodes is associated with a filter arranged to filter the first analog electric potential picked up by this first electrode. Each of the filters may comprise an analog smoothing filter and/or a protection filter against electrostatic discharges.

Advantageously, the first acquisition device comprises a plurality of second electrodes each intended to be connected to one of said separate plants and the multi-channel analog-digital converter comprises a plurality of second inputs each connected to one of the second electrodes. Where applicable, the system is a system for the electrophysiological analysis of a plurality of plants, each plant being connected to a pair of first and second electrodes. According to one example, the pair of first and second electrodes can be connected to the plant so as to measure a linear electric field generated by the superposition of the transient depolarizations of the membranes of the leaf cells and/or by the ion exchanges at the root system. For example, each of the first and second electrodes can be connected to the plant stem to contact the phloem, or alternatively one of the electrodes can be connected to the plant stem and the other of the electrodes can be connected to an underground bulb of the plant.

As a variant, the system is a system for the electrophysiological analysis of a single plant and the first acquisition device comprises a single second electrode intended to be connected to said single plant and connected to each of the second inputs. In this variant, it is thus possible to obtain information relating to the physiological state of the plant at different sections of this plant.

In one embodiment, the or each second electrode is intended to pick up a second analog electric potential, each of the second analog electric potentials received at one of the second inputs of the multi-channel analog-digital converter forming a reference electric potential. If necessary, the multi-channel analog-digital converter is arranged to measure the differences in electric potential between each first and second analog electric potential picked up by each pair of first and second electrodes connected to each of the plants. In the variant of analysis of a single plant, the multi-channel analog-digital converter is arranged to measure the differences in electric potential between each first analog electric potential picked up by each first electrode and the second analog electric potential picked up by the second electrode.

In another embodiment, each second input of the multi-channel analog-digital converter is also connected to a common reference electric potential. Owing to this feature, the common reference electric potential is reinjected via the second electrode(s) into the or each plant, so that the differences in electric potential obtained via the first electric potentials picked up by the first electrodes are located on the same reference frame. For example, each second input of the multi-channel analog-digital converter can be connected to a ground of the first acquisition system, the common reference electric potential being zero.

In another embodiment, the first acquisition device comprises a bias circuit arranged to supply said common reference electric potential, the value of said common reference electric potential being determined from the potential differences measured by the multi-channel analog-digital converter. For example, the bias circuit can be arranged to provide a common reference electric potential, the value of which depends on the average of the electric potential differences measured by the analog-digital converter. This feature in particular allows electric potential differences to be obtained whose values are independent of a phenomenon impacting all the plants connected to the electrodes of the electrophysiological analysis system.

Advantageously, the system can comprise a second acquisition device comprising a plurality of first electrodes each intended to be connected to the same plant or to a separate plant in order to pick up a first analog electric potential and a multi-channel analog-digital converter provided with a plurality of first inputs each connected to one of the first electrodes of the second acquisition system to receive one of the first analog electric potentials. Where appropriate, the first acquisition device may comprise a bias output connected to the bias circuit and the second acquisition device may comprise a bias input connected to said bias output to receive said common reference electric potential, the multi-channel analog-digital converter of the second acquisition device being arranged to measure the potential difference between each first analog electric potential received and said common reference electric potential and to convert each measured potential difference into a digital signal.

In one embodiment of the invention, the multi-channel analog-digital converter of the first acquisition device comprises a plurality of operational amplifiers, one of the inputs of each operational amplifier being connected to one of the first inputs of the multi-channel analog-digital converter to receive a first analog electric potential and the other of the inputs being arranged to receive a reference electric potential, the multi-channel analog-digital converter comprising a plurality of elementary delta-sigma analog-digital converters, each elementary delta-sigma analog-digital converter being connected to the output of one of the operational amplifiers. Where appropriate, the other of the inputs of each operational amplifier is connected to one of the second inputs of the multi-channel digital-analog converter, and in particular to the second input connected to the second electrode of the pair of electrodes to which the first electrode belongs that is connected to the first input of said operational amplifier. The term “connection between the output of one of the operational amplifiers and an elementary delta-sigma analog-digital converter” is understood to mean both a simple connection between an amplifier with a single output and the converter on which the signal amplified by the amplifier passes, and a double connection between a differential output amplifier and the converter where the amplified signal is obtained by the difference between the signals transiting on each of the lines of the double connection.

For example, each operational amplifier to be a Programmable Gain Amplifier (PGA) of the low-noise type. Where appropriate, each operational amplifier may be arranged to have a gain of at most 24 and to introduce noise whose average level is less than 5 μV, and in particular substantially 0.1 μV in the case of an amplifier with a gain of 24. Advantageously, each elementary delta-sigma analog-digital converter is arranged to have a sampling rate greater than 50 SPS (sample per second) or 50 Hz, or even greater than 250 SPS or 250 Hz, and to convert a signal received at input into a 24-bit coded digital signal. If necessary, the unitary assembly formed by an operational amplifier and an elementary delta-sigma analog-digital converter is arranged to have an Effective Number Of Bits (ENOB) of at least 16, and in particular at least 19 for an amplifier gain of 24 and for a sampling rate of 250 SPS. This combination is particularly suitable for the electrophysiological analysis of plants insofar as the electric potential differences resulting from the operational amplifiers are low-amplitude analog signals (of the order of a microvolt) that can amplified by the amplifier while introducing little noise and distortion, owing to a low gain, and that nevertheless be converted by the converter into high-resolution digital signals (of the order of a nanovolt).

If desired, the sampling rate of the multi-channel analog-digital converter, and in particular that of each of the elementary delta-sigma analog-digital converters, can be modulated. This feature makes it possible in particular to adapt the quantity of information collected to a particular type of use, certain uses requiring a lower resolution than others, the reduced sampling rate allowing energy savings and a reduced data flow.

If desired, the multi-channel analog-digital converter can be arranged to verify that a connection is established between each first electrode and the plant. For example, the multi-channel analog-digital converter can be arranged to inject, for example periodically, an excitation current on each of the first electrodes and to conduct a comparison of the signal obtained at the output of the operational amplifier connected to this first electrode with a predetermined threshold so as to determine whether said first electrode is disconnected.

Advantageously, the electrophysiological analysis system comprises a module for digital processing of the digital signals converted by the multi-channel analog-digital converter of the first acquisition device. This digital processing module can for example be arranged to determine a physiological state of the plant(s) connected to the first electrodes by means of the digital signals converted by the multi-channel analog-digital converter. If desired, the digital processing module can be embedded in the first acquisition device, for example by being arranged in the casing. As a variant, the digital processing module can be remote from the first acquisition device. If necessary, the first acquisition device may comprise a memory arranged in the casing, the analog-digital converter comprising a data output connected to said memory, for example via a microcontroller arranged to control the reading and writing of data in said memory, so as to be able to store the converted digital signals in said memory. Alternatively or cumulatively, the first acquisition device may comprise a connector and/or a wireless communication module connected to said data output, for example via a microcontroller arranged to control the shaping and transmission of data via this output, to transmit the converted digital signals to the data processing module.

Advantageously, the processing module can be arranged to implement one or more methods for processing converted digital signals to determine a physiological state of the plant(s). It has in fact been observed that the potential differences thus measured, and therefore the converted digital signals, present variations that are functions of the physiological state of the plant, and in particular of its sunshine, its water stress, its heat stress, etc. These variations are variations of certain frequency components of these converted digital signals, and in particular low-amplitude variations of high-frequency components.

The processing module may be arranged to carry out a spectral analysis of the digital signals. For example, the processing module could be arranged to obtain a spectrum from the digital signals (for example by means of a Fourier transform or by wavelet decomposition) and to determine one or more spectral power indicators from this spectrum, and in particular one or more indicators chosen from the following indicators:
a. a Spectral Edge Frequency (SEF), namely a frequency below which 95% of the total spectral power is concentrated;
b. a Median Edge Frequency (MEF), namely a frequency below which 50% of the total spectral power is concentrated;
c. a power index, namely the total spectral power contained in a given frequency band, in particular a power index in the beta (>0.5 Hz), alpha (0.3-0.4 Hz), theta (0.1-0.2 Hz) and delta (0-0.1 Hz) frequency bands;
d. power index ratios, and in particular a ratio of the delta power index and the theta power index;
e. a Power Spectrum Entropy (PSE).

Advantageously, the processing module will be able to implement methods for analyzing the variations of this or these indicator(s), in particular to recognize a shift in the spectral edge frequency and/or in the median edge frequency according to a given model or even a variation of a power index or a power index ratio according to a given model, or else a variation of the power spectrum entropy according to a given model. It could for example be a method of classifying a variation of this or these indicator(s) from among a library of predetermined variation patterns, each predetermined variation pattern being associated with a given physiological state of a plant. Such a classification method could for example be implemented by a pattern recognition algorithm based on pattern recognition automatic learning from said pattern library.

Advantageously, the system may comprise a plurality of acquisition devices according to the one previously described, the processing module being common to all these acquisition devices.

The invention also relates to a method for the electrophysiological analysis of one or more plants, implemented by a system according to one of the preceding claims.

Advantageously, the method comprises a prior step of connecting the first electrodes, and where applicable the second electrodes, of the first acquisition device of the system to said plant(s).

The present invention is now described using examples, which are only illustrative and in no way limit the scope of the invention, and the accompanying illustrations, wherein:

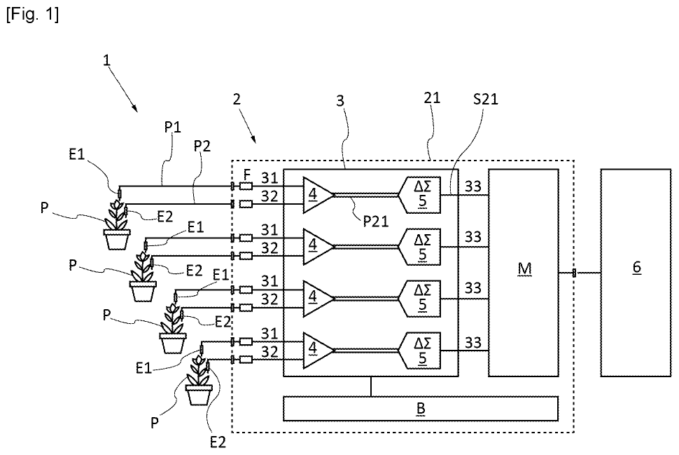

FIG. 1 shows an electrophysiological analysis system according to a first embodiment of the invention;

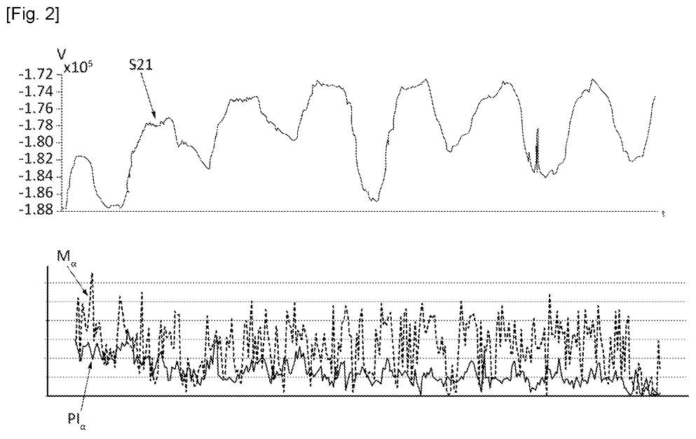

FIG. 2 shows an example of a converted digital signal obtained by means of the system of FIG. 1 as well as an example of an indicator of the physiological state of a plant obtained by means of this converted digital signal;

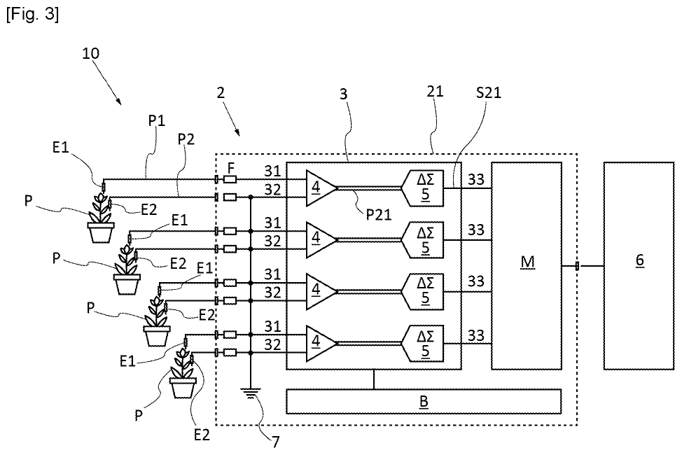

FIG. 3 shows an electrophysiological analysis system according to a second embodiment of the invention;

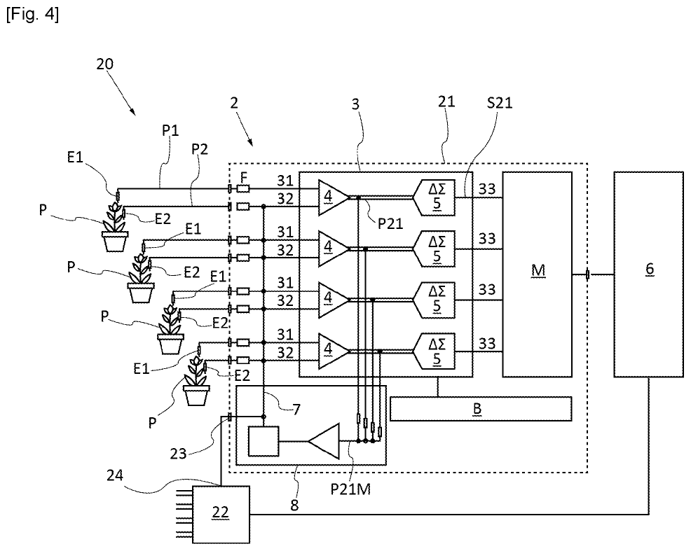

FIG. 4 shows an electrophysiological analysis system according to a third embodiment of the invention; and

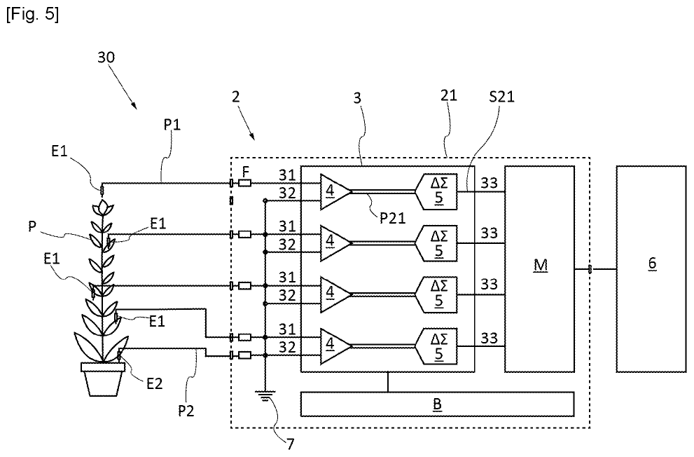

FIG. 5 shows an electrophysiological analysis system according to a fourth embodiment of the invention.

In the following description, identical elements, by structure or by function, appearing in different figures retain the same references, unless otherwise specified.

FIG. 1 schematically and partially shows an electrophysiological analysis system 1 for a plurality of plants P according to a first embodiment of the invention.

The system 1 comprises a first acquisition device 2 comprising a plurality of first electrodes E1 and a plurality of second electrodes E2, each electrode E1 or E2 being a needle-type electrode. Each plant P is thus connected to a pair of electrodes E1 and E2, the first electrode E1 for example being connected at an upper section of the stem of the plant P to be in contact with the phloem and the second electrode E2 for example being connected at a lower section of the stem of the plant P also to be in contact with the phloem.

In this embodiment, each of the first electrodes E1 picks up a first analog electric potential P1 and each of the second electrodes E2 picks up a second analog electric potential P2.

The first acquisition device 2 comprises a casing 21 in which are arranged a multi-channel analog-digital converter 3, an electric power source B and a memory M.

The converter 3 is provided with a plurality of first inputs 31, each of the first inputs 31 being connected to one of the first electrodes E1, via a connector made in the casing 21, so that each first electric potential P1 picked up by one of the first electrodes E1 is simultaneously received by the converter 3. Equivalently, the converter 3 is provided with a plurality of second inputs 32 connected to the second electrodes E2 to simultaneously receive the second electric potentials P2.

Each of the inputs 31 and 32 is associated with a filter F arranged to filter the first or the second electric potential P1, P2 intended to be received by this input.

The converter 3 comprises a plurality of operational amplifiers 4. One of the inputs of each amplifier 4 is connected to one of the first inputs 31 linked to a first electrode E1 of a given pair of electrodes, and the other of the inputs is thus connected to one of the second inputs 32 linked to the second electrode E2 of this pair of given electrodes. Each amplifier 4 thus receives the first and second analog electric potentials P1 and P2 picked up by the pair of electrodes E1 and E2 connected to one of the plants P.

In the example shown, each second analog electric potential P2 emitted by one of the plants P forms a reference potential with which the first analog electric potential P1 emitted by this plant P is compared by one of the amplifiers 4. Each amplifier 4 thus measures the difference in electric potential P21 between the first and second electric potentials P1 and P2 picked up by the pair of electrodes E1 and E2 connected to one of the plants P. In the example described, each operational amplifier is a low-noise Programmable Gain Amplifier (PGA) whose gain can be programmed up to a value of 24 without an average noise level introduced into the signal amplified by the amplifier exceeding a value of substantially 2 μV for this gain of 24.

The converter 3 also comprises a plurality of elementary analog-digital delta-sigma converters 5, each elementary converter 5 being connected to the differential output of one of the amplifiers 4 to receive the electric potential difference P21 measured by this amplifier 4 and to convert it into a digital signal S21. In the example described, each delta-sigma elementary converter 5 is arranged to sample the electric potential difference P21, for example at a sampling frequency of 250 Hz, so as to obtain a digital signal S21 encoded on 24 bits. This combination of low-noise PGAs and delta-sigma-type converters results in an effective number of bits of at least 19 when the PGA gain is set to 24. In this way, the digital signal S21 obtained at the output of the elementary converter 5 has a particularly satisfactory resolution, given the voltage levels observed for the electric potentials picked up by the electrodes. It should be noted that the sampling frequency can advantageously be modulated, in particular according to the desired recording duration.

In the example described, the amplifiers 4 and the elementary converters 5 are integrated on the same integrated circuit, powered by the electric power source B.

The converter 3 comprises data outputs 33 for transmitting the converted digital signals S21 to the memory M in which these signals S21 are stored. It should be noted that the writing of data from the output 33 to the memory M is performed by a microcontroller (not shown).

The system 1 comprises a processing module 6, remote from the acquisition device 2 and its casing 21, arranged to digitally process the converted digital signals S21 in order to determine a physiological state of the plants P. In the example described, the casing 21 comprises a connector connected to the memory 5, via said microcontroller (not shown), to transmit the converted digital signals S21 to the processing module 6.

FIG. 2 shows an example of a converted digital signal S21 obtained by the acquisition device 2 for one of the plants P during an acquisition period of 7 days. In addition, also shown below is an indicator PI_{α} determined by the processing module 6 from the digital signal S21 shown above. This indicator corresponds to the evolution over time during this acquisition period of a power index corresponding to the total spectral power of the digital signal S21 in the frequency band from 0.3 Hz to 0.4 Hz. Also shown on the same graph, in dotted lines, is a model M_{α} of the evolution of the same power index for a plant of the same species that is irrigated. As can be seen, the variation of the indicator PI_{α} is different from that of the model M_{α}. In particular, this indicator PI_{α} does not extend above a threshold value of 0.2. Therefore, it is possible to carry out an analysis of the variation of this indicator PI_{α} to deduce the physiological state of the plant P, and thus to determine that this plant P is under water stress and that it is necessary to irrigate it.

Although the example described in FIG. 1 only describes a single acquisition device 2, it goes without saying that the analysis system 1 could use a plurality of acquisition devices, each being similar to the first acquisition device 2 and being connected to another plurality of plants, the digital signals converted by these devices all being transmitted to the processing module 6 to be processed there.

FIG. 3 schematically and partially shows an electrophysiological analysis system 10 for a plurality of plants P according to a second embodiment of the invention.

This system 10 is structurally and functionally similar to the system 1 described in FIG. 1, with the exception of the features described below.

In the embodiment of FIG. 3, each of the second inputs 32 of the converter 3 is connected to the second electrodes E2 and to a common reference electric potential 7, in this case a ground of the casing 21. The values of the second electric potentials P2 are therefore all equal to the common reference electric potential 7, in this case to a zero value. In addition, the common reference electric potential 7 is thus reinjected into each of the plants P via the second electrodes E2 so that the first potentials P1, and therefore the differences in electric potential P21 measured by the amplifiers 4, are refocused and can thus be acquired according to the measurement range of the system 10. It should be noted that the second inputs in this case are not associated with a filter.

FIG. 4 schematically and partially shows an electrophysiological analysis system 20 for a plurality of plants P according to a third embodiment of the invention.

This system 20 is structurally and functionally similar to the system 10 described in FIG. 3, with the exception of the features described below.

In the embodiment of FIG. 4, the first acquisition device 2 comprises a bias circuit 8, integrated into the casing 21 and arranged to supply said reference electric potential 7.

The bias circuit 8 is designed to obtain an average value P21M of the differences in electric potential P21 measured by each of the amplifiers 4, and to amplify and filter this average value P21M so as to obtain said common reference electric potential 7. Identically to the embodiment of FIG. 3, this common reference electric potential 7 is reinjected into each of the plants P.

Furthermore, in the embodiment of FIG. 4, the system 20 comprises a second acquisition device 22, similar to the first acquisition device 2. The first acquisition device 2 comprises a bias output 23 connected to the bias circuit 8 to receive the common reference electric potential 7. The second acquisition device 22 comprises a bias input 24 connected to the bias output 23, all the second inputs of the converter of the second acquisition device 22 being connected to said bias input 24 so that the amplifiers of the converter of the second acquisition device 22 measure the differences in electric potential between the electric potentials picked up by the first electrodes of the second acquisition device 22 and the common reference electric potential 7 supplied by the bias circuit 8 of the first acquisition device.

FIG. 5 schematically and partially shows an electrophysiological analysis system 30 for a single plant P according to a fourth embodiment of the invention.

This system 40 is structurally and functionally similar to the system 10 described in FIG. 3, with the exception of the features described below.

In the embodiment of FIG. 5, each of the first electrodes E1 is connected to the same plant P, at different sections of this plant. In addition, the first acquisition device 2 comprises a single second electrode E2, also connected to said plant P, and each of the second inputs 32 of the converter 3 is connected to said second electrode E2 and to the common reference electric potential 7. In this way, each amplifier 4 is arranged to measure an electric potential difference between the first electric potential P1 picked up by one of the first electrodes E1 at a given section of the plant P and the common reference electric potential 7. It is thus possible to obtain information relating to the physiological state of the plant P at the different sections and possibly to observe the propagation of a given state in these different sections.

The preceding description clearly explains how the invention makes it possible to achieve the objectives it has set itself, and in particular by proposing a system for the electrophysiological analysis of one or more plants using a multi-channel analog-digital converter integrated into a casing. It is thus understood that owing to the multi-channel analog-digital converter, it is possible to simultaneously pick up a plurality of electrical signals emitted by the plant(s) and to determine, from these signals, differences in electric potential, which are converted into digital signals that can be analyzed, immediately or later. In addition, the integration of the multi-channel analog-digital converter into a casing, further integrating other components such as an electric power source and a memory, makes it possible to make the analysis system autonomous and resistant to the various environmental constraints of the agricultural environment.

In any event, the invention cannot be limited to the embodiments specifically described in this document, and extends in particular to all equivalent means and to any technically operative combination of these means. In particular, other modes of power supply for the multi-channel analog-digital converter may be provided, and in particular electric power supply by an external power source. Furthermore, provision may be made to integrate the digital processing module into the casing of the acquisition device, in particular when the analysis system is associated with a control unit for at least one environmental factor capable of modifying the physiological state of the plant(s), for example such as sunshine, irrigation or temperature, the control unit being arranged to control and modify said factor as a function of the physiological state determined by the digital processing module from the converted digital signals.

1. A system (1, 10, 20, 30) for the electrophysiological analysis of one or more plants (P), comprising a first acquisition device (2) comprising a plurality of first electrodes (E1) each intended to be connected to the same plant or to a separate plant to pick up a first analog electric potential (P1), the first acquisition device comprising a casing (21) in which a multi-channel analog-digital converter (3) is arranged that is provided with a plurality of first inputs (31) each connected to one of the first electrodes to receive one of the first analog electric potentials, the multi-channel analog-digital converter being arranged to measure the potential difference (P21) between each first analog electrical potential received and a reference electric potential (P2, 7) and to convert each measured potential difference into a digital signal (S21).

2. The system (1, 10, 20, 20) according claim 1, wherein the first acquisition device (2) comprises at least one electric power source (B) arranged to power the multi-channel analog-digital converter (3).

3. The system (1, 10, 20) according to claim 1, wherein the first acquisition device (2) comprises a plurality of second electrodes (E2) each intended to be connected to one of said separate plants (P) and the multi-channel analog-digital converter (3) comprises a plurality of second inputs (32) each connected to one of the second electrodes.

4. The system (1) according to claim 3, wherein each second electrode (E2) is intended to pick up a second analog electric potential (P2), each of the second analog electric potentials received at one of the second inputs (32) of the multi-channel analog-digital converter (3) forming a reference electric potential.

5. The system (10, 20) according to claim 3, wherein each second input (32) of the multi-channel analog-digital converter (3) is also connected to a common reference electric potential (7).

6. The system (20) according to claim 5, wherein the first acquisition device (2) comprises a bias circuit (8) arranged to determine said common reference electric potential (7) from the potential differences (P21) measured by the multi-channel analog-digital converter (3).

7. The system (20) according to claim 1, the system comprising a second acquisition device (22) comprising a plurality of first electrodes each intended to be connected to the same plant or to a separate plant in order to pick up a first analog electric potential and a multichannel analog-digital converter provided with a plurality of first inputs each connected to one of the first electrodes of the second acquisition system to receive one of the first analog electric potentials, in which the first acquisition device (2) comprises a bias output (23) connected to the bias circuit (8) and the second acquisition device comprises a bias input (24) connected to said bias output to receive said common reference electric potential (7), the multi-channel analog-digital converter of the second acquisition device being arranged to measure the potential difference between each first received analog electric potential and said common reference electric potential and to convert each measured potential difference into a digital signal.

8. The system (1, 10, 20, 30) according to claim 1, wherein the multi-channel analog-digital converter (3) of the first acquisition device (2) comprises a plurality of operational amplifiers (4), one of the inputs of each operational amplifier being connected to one of the first inputs (31) of the multi-channel analog-digital converter to receive a first analog electric potential (P1) and the other of the inputs being arranged to receive a reference electric potential (P2, 7), the multi-channel analog-digital converter comprising a plurality of elementary delta-sigma analog-digital converters (5), each elementary delta-sigma analog-digital converter being connected to the output of one of the operational amplifiers.

9. The system (1, 10, 20, 30) according to claim 1, comprising a module (6) for digital processing of the digital signals (S21) converted by the multi-channel analog-digital converter (3) of the first acquisition device (2).

10. A method for the electrophysiological analysis of one or more plants, implemented by a system (1, 10, 20, 30) according to claim 1.
